(19) United States
(12) Patent Application Publication
(10) Pub. No.: US 2009/0163726 A1
(43) Pub. Date: Jun. 25, 2009

(54) CATALYST SYSTEM FOR PREPARING CARBOXYLIC ACIDS AND/OR CARBOXYLIC ANHYDRIDES

(75) Inventors: Hagen Wilmer, Ludwigshafen (DE); Cornelia Dobner, Ludwigshafen (DE); Frank Rosowski, Mannheim (DE); Sebastian Storck, Mannheim (DE); Jürgen Zühlke, Speyer (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/301,420

(22) PCT Filed: May 14, 2007
(86) PCT No.: PCT/EP2007/054621

§ 371 Date: Nov. 18, 2008

(30) Foreign Application Priority Data
May 19, 2006 (EP) 06114230.3

(51) Int. Cl. C07D 307/77; B01J 23/04; B01J 27/198

(52) U.S. Cl. 549/230; 502/344; 502/209

(57) ABSTRACT

The present invention relates to a catalyst system for preparing carboxylic acids and/or carboxylic anhydrides which has at least four catalyst layers arranged one on top of another in the reaction tube, the ratio of the bed lengths of the more selective catalyst layers to the bed lengths of the more active catalyst layers being between 1.4 and 2. The present invention further relates to a process for gas phase oxidation in which a gaseous stream which comprises a hydrocarbon and molecular oxygen is passed through a plurality of catalyst layers, the ratio of the bed lengths of the more selective catalyst layers to the bed lengths of the more active catalyst layers being between 1.4 and 2.

The present invention relates to a catalyst system for preparing carboxylic acids and/or carboxylic anhydrides which has at least four catalyst layers arranged one on top of another in the reaction tube, the ratio of the bed lengths of the more selective catalyst layers to the bed lengths of the more active catalyst layers being between 1.4 and 2. The present invention further relates to a process for gas phase oxidation in which a gaseous stream which comprises a hydrocarbon and molecular oxygen is passed through a plurality of catalyst layers, the ratio of the bed lengths of the more selective catalyst layers to the bed lengths of the more active catalyst layers being between 1.4 and 2.

A multitude of carboxylic acids and/or carboxylic anhydrides is prepared industrially by the catalytic gas phase oxidation of hydrocarbons, such as benzene, the xylenes, naphthalene, toluene or durene, in fixed bed reactors. In this way, it is possible to obtain, for example, benzoic acid, maleic anhydride, phthalic anhydride, isophthalic acid, terephthalic acid or pyromellitic anhydride. In general, a mixture of an oxygenous gas and the starting material to be oxidized is passed through tubes in which a bed of a catalyst is disposed. For temperature regulation, the tubes are surrounded by a heat carrier medium, for example a salt melt.

Even though the excess heat of reaction is removed by the heat carrier medium, local temperature maxima (hotspots) form in the catalyst bed, in which there is a higher temperature than in the remaining part of the catalyst bed or than in the remaining part of the catalyst layer. These hotspots lead to side reactions, for example the total combustion of the starting material, and can damage the catalyst irreversibly from a certain hotspot temperature.

To attenuate these hotspots, various measures have been taken. In particular, as described in DE-A 40 13 051, there has been a transition to the arrangement of catalysts of different activity layer by layer in the catalyst bed, the more selective catalyst generally being arranged toward the gas inlet and the more active catalyst toward the gas outlet.

The activity of the catalysts or catalyst systems used for gas phase oxidation accordingly decreases with increasing operating time. A proportion of unconverted hydrocarbons or partly oxidized intermediates which Increases with operating time gets into regions of the catalyst bed further downstream, i.e. into more active catalyst layers, so that the reaction increasingly shifts toward the reactor outlet and the hotspot migrates downstream. Since downstream catalyst layers are generally more active but less selective, undesired overoxidation and other side reactions increase. The two effects mentioned have the effect that the product yield falls with operating time.

Experience has shown that the catalysts mentioned have a lifetime of from 2 to 5 operating years, after which their effectiveness, both with regard to the conversion and to the selectivity, declines to such an extent that further use is no longer economic.

CN 1616148 describes a catalyst system for the preparation of phthalic anhydride. The catalysts are composed mainly of V₂O₅, TiO₂, Sb₂O₃ and Cs. The catalyst system is divided into four catalyst layers. The first, uppermost, most selective catalyst layer has a length of from 0.9 to 1.5 m; the second catalyst layer has a length of from 0.2 to 1.0 m; the third catalyst layer has a length of from 0.2 to 1.0 m and the last, most active catalyst layer has a length of from 0.2 to 1.0 m. The ratio of the bed lengths of the more selective catalyst layers to the bed lengths of the more active catalyst layers is thus between 0.5 and 6.25. In the example, a catalyst system with the bed length 135/55/80/80 cm is disclosed. The ratio of the bed lengths of the more selective catalyst layers to the bed lengths of the more active catalyst layers is 1.18. The initial yield of phthalic anhydride was 114%. At a loading of 90 g, a yield of >113% was still achievable after one year.

WO 04/103944 describes a process for preparing phthalic anhydride in which catalyst systems which have four catalyst layers are used, the first, most selective catalyst layer making up from 27 to 55% of the total bed length of the catalyst, the second catalyst layer from 5 to 22%, the third catalyst layer from 8 to 35% and the fourth catalyst layer from 16 to 40%. The ratio of the bed lengths of the more selective catalyst layers to the bed lengths of the more active catalyst layers is thus between 0.4 and 3.5. In the example, a catalyst system with the bed length 130/50/70/70 cm is disclosed. The ratio of the bed lengths of the more selective catalyst layers to the bed lengths of the more active catalyst layers is 1.3. The initial yield of phthalic anhydride after 50 days at a loading of 100 g/m³ (STP) of o-xylene is 113.5%. A long-term test is not disclosed.

In spite of the advances achieved in the prior art relating to a long-lasting high yield of phthalic anhydride at high o-xylene loading, there is a constant need for optimization with regard to an improved or constant high yield with high or increased hydrocarbon loading.

It is thus an object of the invention to provide a catalyst system for gas phase oxidation which has very uniform thermal stress on the catalyst system. It is thus a further object of the invention to provide a catalyst system for gas phase oxidation, which also affords a high yield of carboxylic acids and/or carboxylic anhydrides with a high loading of hydrocarbons over a long period.

The object is achieved by a catalyst system for preparing carboxylic acids and/or carboxylic anhydrides which has at least four catalyst layers arranged one on top of another in the reaction tube, the ratio of the bed lengths of the more selective catalyst layers to the bed lengths of the more active catalyst layers being between 1.4 and 2.

Advantageously, the ratio of the bed lengths of the more selective catalyst layers to the bed lengths of the more active catalyst layers is between 1.4 and 2, preferably between 1.5 and 1.8, in particular between 1.6 and 1.7.

The total length of the catalyst bed is typically from 2.5 to 4 m, preferably from 2.8 to 3.4 m.

In the present invention, the activity of a catalyst layer is defined as follows: the higher the conversion for a specific reactant mixture at the same salt bath temperature, the higher its activity.

Moreover, in the present invention, the selectivity of a catalyst layer is defined as follows: the higher the yield of a specific product at the same salt bath temperature, the higher its selectivity.

The activity/selectivity of the catalysts can be varied, for example, by adding promoters to the active composition, by adjusting the BET surface area of the catalysts, by virtue of the active composition content, i.e. by virtue of the active composition per tube volume, by virtue of the empty space between the individual shaped catalyst bodies or by virtue of the content of inert substances.

Measures for controlling the activity of gas phase oxidation catalysts based on vanadium oxide and titanium dioxide are known per se to those skilled in the art and are described, for example, in European patent application 06112510.0 on pages 3 and 4.

The catalytically active composition of all catalysts preferably comprises at least vanadium oxide and titanium dioxide. The BET surface area of the catalytically active components of the catalyst is advantageously in the range from 5 to 50 m²/g, preferably from 5 to 40 m²/g, in particular from 9 to 35 m²/g. The active composition content is preferably from 3 to 15% by weight, in particular from 4 to 12% by weight, based on the overall catalyst composition.

Advantageously, the activity is controlled with the aid of the cesium promoter. Active catalyst layers typically have a cesium content of ≦0.1% by weight based on the active composition content. Selective catalyst layers typically have a cesium content of >0.1% by weight based on the active composition content.

In a four-layer catalyst system, the second selective layer is advantageously longer than the third and/or fourth catalyst layer. The second catalyst layer is preferably at least 5% longer, especially at least 10% longer, than the third and/or fourth catalyst layer.

In a four-layer catalyst system, the first, most selective catalyst layer advantageously has a length of from 30 to 50% of the overall catalyst bed, preferably from 35 to 45%; the second catalyst layer advantageously has from 18 to 25% of the overall catalyst bed, preferably from 20 to 23%; and the third and fourth catalyst layer advantageously each have from 15 to 22% of the overall catalyst bed, preferably from 17 to 20%, the second catalyst layer more preferably having a longer bed length than the third and/or fourth catalyst layer.

In a four-layer catalyst system, the ratio of the first catalyst layer to the second catalyst layer is advantageously less than 2.4, preferably between 1.6 and 2.2, in particular between 1.8 and 2.0.

The catalysts used in the process according to the invention are generally coated catalysts in which the catalytically active composition is applied in coating form on an inert support. The layer thickness of the catalytically active composition is generally from 0.02 to 0.25 mm, preferably from 0.05 to 0.15 mm. In general, the catalysts have an active composition layer applied in coating form with essentially homogeneous chemical composition. In addition, it is also possible for two or more different active composition layers to be applied successively to a support. Reference is then made to a two-layer or multilayer catalyst (see, for example, DE 19839001 A1).

The inert support materials used may be virtually all prior art support materials, as are used advantageously in the preparation of coated catalysts for the oxidation of aromatic hydrocarbons to aldehydes, carboxylic acids and/or carboxylic anhydrides, as described, for example, in WO 2004/103561 on pages 5 and 6. Preference is given to using steatite in the form of spheres having a diameter of from 3 to 6 mm or of rings having an external diameter of from 5 to 9 mm, a length of from 4 to 7 mm and an internal diameter of from 3 to 7 mm.

The individual layers of the coated catalyst can be applied by any methods known per se, for example by spray application of solutions or suspensions in a coating drum, or coating with a solution or suspension in a fluidized bed, as described, for example, in WO 2005/030388, DE 4006935 A1, DE 19824532 A1, EP 0966324 B1.

The activity of the catalyst layers advantageously increases from the uppermost catalyst layer (gas inlet side) to the lowermost catalyst layer (gas outlet side). If appropriate, it would be possible to use upstream or intermediate catalysts with a higher activity (European patent application 06112510.0) or one or more moderators (European patent application 06008816.8). The activity of the catalyst layers preferably increases continuously from the gas inlet side to the gas outlet side.

In a preferred embodiment of a four-layer catalyst system for preparing phthalic anhydride,

a) the least active catalyst, on nonporous and/or porous support material, has from 7 to 11% by weight, based on the overall catalyst, of active composition comprising from 4 to 11 % by weight of V₂O₅, from 0 to 4% by weight of Sb₂O₃ or Nb₂O₅, from 0 to 0.5% by weight of P, from 0.1 to 1.1% by weight of alkali metal and, as the remainder, TiO₂ in anatase form,

b) the next catalyst arranged in flow direction, on nonporous and/or porous support material, has from 7 to 12% by weight, based on the overall catalyst, of active composition comprising from 4 to 15% by weight of V₂O₅, from 0 to 4% by weight of Sb₂O₃ or Nb₂O₅, from 0 to 0.5% by weight of P, from 0.1 to 1% by weight of alkali metal and, as the remainder, TiO₂ in anatase form,

c) the next catalyst arranged in flow direction, on nonporous and/or porous support material, has from 7 to 12% by weight, based on the overall catalyst, of active composition comprising from 5 to 13% by weight of V₂O₅, from 0 to 4% by weight of Sb₂O₃ or Nb₂O₅, from 0 to 0.5% by weight of P, from 0 to 0.4% by weight of alkali metal and, as the remainder, TiO₂ in anatase form,

d) and the next catalyst arranged in flow direction, on nonporous and/or porous support material, has from 8 to 12% by weight, based on the overall catalyst, of active composition comprising from 10 to 30% by weight of V₂O₅, from 0 to 4% by weight of Sb₂O₃ or Nb₂O₅, from 0 to 0.5% by weight of P, from 0 to 0. 1% by weight of alkali metal and, as the remainder, TiO₂ in anatase form,

the alkali metal used preferably being cesium.

The present invention further relates to a process for gas phase oxidation in which a gaseous stream which comprises at least one hydrocarbon and molecular oxygen is passed through at least four catalyst layers arranged one on top of another in a reaction tube, the ratio of the bed lengths of the more selective catalyst layers to the bed lengths of the more active catalyst layers being between 1.4 and 2.

The process according to the invention is advantageously suitable for the gas phase oxidation of aromatic C₆- to C₁₀-hydrocarbons, such as benzene, the xylenes, toluene, naphthalene or durene (1,2,4,5-tetramethylbenzene), to carboxylic acids and/or carboxylic anhydrides, such as maleic anhydride, phthalic anhydride, benzoic acid and/or pyromellitic dianhydride.

The process is suitable particularly for preparing phthalic anhydride from o-xylene and/or naphthalene. The gas phase reactions for preparing phthalic anhydride are common knowledge and are described, for example, in WO 2004/103561 on page 6.

The present invention can achieve longer catalyst lifetimes. Even at high loadings of 100 g/m³ (STP) of o-xylene, a yield of over 113% is still achieved after one year. This corresponds to a capacity increase over CN 1616148, in which the reaction was performed only at 90 g/m³ (STP) of o-xylene loading, of over 10 percent.

### EXAMPLES

### Preparation of the Catalysts

### Catalyst KL1

The catalysts are prepared as described in European patent application 06112510.0 in the examples.

After calcination of the catalyst at 450° C. for one hour, the active composition applied to the steatite rings was 8.0%. The analyzed composition of the active composition consisted of 7.1% V₂O₅, 1.8% Sb₂O₃, 0.36% Cs, remainder TiO₂.

### Catalyst KL2

Preparation analogous to KL1 with variation of the composition of the suspension. After calcination of the catalyst at 450° C. for one hour, the active composition applied to the steatite rings was 8.0%. The analyzed composition of the active composition consisted of 7.1% V₂O₅, 2.4% Sb₂O₃, 0.24% Cs, remainder TiO₂.

### Catalyst KL3

Preparation analogous to KL1 with variation of the composition of the suspension. After calcination of the catalyst at 450° C. for one hour, the active composition applied to the steatite rings was 8.5%. The analyzed composition of the active composition consisted of 7.1% V₂O₅, 2.4% Sb₂O₃, 0.1% Cs, remainder TiO₂.

### Catalyst KL4

Preparation analogous to KL1 with variation of the composition of the suspension. After calcination of the catalyst at 450° C. for one hour, the active composition applied to the steatite rings was 9.0%. The analyzed composition of the active composition consisted of 20% V₂O₅, 0.38% P, remainder TiO₂.

### Processes For Preparing Phthalic Anhydride

### A) Inventive

The catalysts were introduced into a reaction tube of internal diameter 25 mm. Starting from the reactor inlet, the catalyst bed had the following composition: KL1/KL2/KL3/KL4=130/70/60/60 cm.

### B) Noninventive

The catalysts were introduced into a reaction tube of internal diameter 25 mm. Starting from the reactor inlet, the catalyst bed had the following composition: KL1/KL2/KL3/KL4=130/50/70/70 cm.

### Catalaytic Results

At the same volume flow rate (4 m³/h (STP)/h), after running-up to 100 g/m³ (STP), the following results were achieved:

| | Salt bath | | |
|---|---|---|---|
| | temperature | PA yield in m/m % | PA yield in m/m % |
| Catalyst | in ° C. | (after 30 days) | (after 300 days) |
| A (inventive) | 356 | 114.6 | 113.1 |
| B (non inven- | 355 | 114.4 | 112.2 |
| tive) | | | |

1. A catalyst system for preparing carboxylic acids and/or carboxylic anhydrides which has at least four catalyst layers arranged one on top of another in the reaction tube, the ratio of the bed lengths of the more selective catalyst layers to the bed lengths of the more active catalyst layers being between 1.4 and 2.

2. The catalyst system according to claim 1, wherein the ratio of the bed lengths of the more selective catalyst layers to the bed lengths of the more active catalyst layers is between 1.5 and 1.8.

3. The catalyst system according to claim 1, wherein the total bed length of the catalyst system is from 2.5 to 4 m.

4. The catalyst system according to claim 1, wherein the active catalyst layers have a cesium content of ≦0.1 % by weight based on the active composition content.

5. The catalyst system according to claim 1, wherein, in a four-layer catalyst system, the second catalyst layer viewed from the gas inlet is longer than the third and/or fourth catalyst layer.

6. The catalyst system according to claim 5, wherein the first catalyst layer has a length of from 30 to 50% of the overall catalyst bed, the second catalyst layer from 18 to 25% of the overall catalyst bed, and the third and the fourth catalyst layer in each case from 15 to 22% by weight of the overall catalyst bed.

7. The catalyst system according to claim 5, wherein the ratio of the bed length of the first catalyst layer to the second catalyst layer is less than 2.4.

8. The catalyst system according to claim 5 which has four catalyst layers arranged one on top of another, wherein
a) the least active catalyst, on nonporous and/or porous support material, has from 7 to 11% by weight, based on the overall catalyst, of active composition comprising from 4 to 11% by weight of V₂O₅, from 0 to 4% by weight of Sb₂O₃ or Nb₂O₅, from 0 to 0.5% by weight of P, from 0.1 to 1.1% by weight of alkali metal and, as the remainder, TiO₂ in anatase form,
b) the next catalyst arranged in flow direction, on nonporous and/or porous support material has from 7 to 12% by weight, based on the overall catalyst, of active composition comprising from 4 to 15% by weight Of V₂O₅, from 0 to 4% by weight of Sb₂O₃ or Nb₂O₅, from 0 to 0.5% by weight of P, from 0.1 to 1% by weight of alkali metal and, as the remainder, TiO₂ in anatase form,
c) the next catalyst arranged in flow direction, on nonporous and/or porous support material has from 7 to 12% by weight, based on the overall catalyst, of active composition comprising from 5 to 13% by weight of V₂O₅, from 0 to 4% by weight of Sb₂O₃ or Nb₂O₅, from 0 to 0.5% by weight of P, from/to 0.4% by weight of alkali metal and, as the remainder, TiO₂ in anatase form,
d) and the next catalyst arranged in flow direction, on nonporous and/or porous support material has from 8 to 12% by weight, based on the overall catalyst, of active composition comprising from 10 to 30% by weight of V₂O₅, from 0 to 4% by weight of Sb₂O₃ or Nb₂O₅, from 0 to 0.5% by weight of P, from 0 to 0. 1% by weight of alkali metal and, as the remainder, TiO₂ in anatase form.

9. A process for gas phase oxidation in which a gaseous stream which comprises at least one hydrocarbon and molecular oxygen is passed through at least four catalyst layers arranged one on top of another in a reaction tube, the ratio of the bed lengths of the more selective catalyst layers to the bed lengths of the more active catalyst layers being between 1.4 and 2.

10. The process according to claim 9 for preparing phthalic anhydride by catalytic gas phase oxidation of xylene and/or naphthalene with a molecular oxygen-comprising gas.

11. The catalyst system according to claim 2, wherein the total bed length of the catalyst system is from 2.5 to 4 m.

12. The catalyst system according to claim 2, wherein the active catalyst layers have a cesium content of ≦0.1% by weight based on the active composition content.

13. The catalyst system according to claim 3, wherein the active catalyst layers have a cesium content of ≦0. 1% by weight based on the active composition content.

14. The catalyst system according to claim 2, wherein, in a four-layer catalyst system, the second catalyst layer viewed from the gas inlet is longer than the third and/or fourth catalyst layer.

15. The catalyst system according to claim 3, wherein, in a four-layer catalyst system, the second catalyst layer viewed from the gas inlet is longer than the third and/or fourth catalyst layer.

16. The catalyst system according to claim 4, wherein, in a four-layer catalyst system, the second catalyst layer viewed from the gas inlet is longer than the third and/or fourth catalyst layer.

17. The catalyst system according to claim 6, wherein the ratio of the bed length of the first catalyst layer to the second catalyst layer is less than 2.4.

18. The catalyst system according to claim 6 which has four catalyst layers arranged one on top of another, wherein
a) the least active catalyst, on nonporous and/or porous support material, has from 7 to 11% by weight, based on the overall catalyst, of active composition comprising from 4 to 11% by weight of V₂O₅, from 0 to 4% by weight of Sb₂O₃ or Nb₂O₅, from 0 to 0.5% by weight of P, from 0.1 to 1.1% by weight of alkali metal and, as the remainder, TiO₂ in anatase form,
b) the next catalyst arranged in flow direction, on nonporous and/or porous support material has from 7 to 12% by weight, based on the overall catalyst, of active composition comprising from 4 to 15% by weight of V₂O₅, from 0 to 4% by weight of Sb₂O₃ or Nb₂O₅, from 0 to 0.5% by weight of P, from 0.1 to 1% by weight of alkali metal and, as the remainder, TiO₂ in anatase form,
c) the next catalyst arranged in flow direction, on nonporous and/or porous support material has from 7 to 12% by weight, based-on the overall catalyst, of active composition comprising from 5 to 13% by weight of V₂O₅, from 0 to 4% by weight of Sb₂O₃ or Nb₂O₅, from 0 to 0.5% by weight of P, from/to 0.4% by weight of alkali metal and, as the remainder, TiO₂ in anatase form,
d) and the next catalyst arranged in flow direction, on nonporous and/or porous support material has from 8 to 12% by weight, based on the overall catalyst, of active composition comprising from 10 to 30% by weight of V₂O₅, from 0 to 4% by weight of Sb₂O₃ or Nb₂O₅, from 0 to 0.5% by weight of P, from 0 to 0.1% by weight of alkali metal and, as the remainder, TiO₂ in anatase form.

19. The catalyst system according to claim 7 which has four catalyst layers arranged one on top of another, wherein
a) the least active catalyst, on nonporous and/or porous support material, has from 7 to 11% by weight, based on the overall catalyst, of active composition comprising from 4 to 11% by weight of V₂O₅, from 0 to 4% by weight of Sb₂O₃ or Nb₂O₅, from 0 to 0.5% by weight of P, from 0.1 to 1.1% by weight of alkali metal and, as the remainder, TiO₂ in anatase form,
b) the next catalyst arranged in flow direction, on nonporous and/or porous support material has from 7 to 12% by weight, based on the overall catalyst, of active composition comprising from 4 to 15% by weight of V₂O₅, from 0 to 4% by weight of Sb₂O₃ or Nb₂O₅, from 0 to 0.5% by weight of P, from 0.1 to 1% by weight of alkali metal and, as the remainder, TiO₂ in anatase form,
c) the next catalyst arranged in flow direction, on nonporous and/or porous support material has from 7 to 12% by weight, based on the overall catalyst, of active composition comprising from 5 to 13% by weight of V₂O₅, from 0 to 4% by weight of Sb₂O₃ or Nb₂O₅, from 0 to 0.5% by weight of P, from/to 0.4% by weight of alkali metal and, as the remainder, TiO₂ in anatase form,
d) and the next catalyst arranged in flow direction, on nonporous and/or porous support material has from 8 to 12% by weight, based on the overall catalyst, of active composition comprising from 10 to 30% by weight of V₂O₅, from 0 to 4% by weight of Sb₂O₃ or Nb₂O₅, from 0 to 0.5% by weight of P, from 0 to 0.1% by weight of alkali metal and, as the remainder, TiO₂ in anatase form.

20. The catalyst system according to claim 11, wherein the active catalyst layers have a cesium content of ≦0. 1% by weight based on the active composition content.
